(19) United States
(12) Patent Application Publication
(10) Pub. No.: US 2012/0283519 A1
(43) Pub. Date: Nov. 8, 2012

(54) Surgical retractor

(76) Inventors: Anthony Hung Nguyen, Santa Clara, CA (US); Yahya Amin Medlij, San Jose, CA (US)

(21) Appl. No.: 13/101,152

(22) Filed: May 5, 2011

(51) Int. Cl. A61B 1/32

(52) U.S. Cl. 600/208

(57) ABSTRACT

A circumferential surgical retractor to hold an incision apart and provide access through its aperture opening. The retractor has no moving parts and relies on its flanges to retract tissues. Selection of different sizes of the retractor permits its use in differing surgical incisions.

### REFERENCES CITED

U.S. PATENT DOCUMENTS

| | | | |
|---|---|---|---|
| 4,984,564 | January 1991 | Yuen | 600/207 |
| 5,159,921 | November 1992 | Hoover | 128/20 |
| 6,162,172 | December 2000 | Cosgrove | 600/208 |
| 7,018,332 | March 2006 | Masson | 600/227 |

### FIELD OF INVENTION

The present invention relates to surgical retractor apparatus and associated methods for retracting a surgical incision and retaining the incision open during surgical procedures.

### BACKGROUND OF THE INVENTION

Self retaining retractors often rely on mechanisms such as scissor action opening, retractor paddles braced to a larger adjustable frame, and/or ratchet mechanism to allow adjustment of the retractor to fit the desired size of the surgical field being exposed. Frequently this leads to a heavy and complex mechanical surgical retractor that may limit its applicability.

Such is the case during surgical procedures where a body part is positioned or suspended to permit easier access as during arthroscopy. Standard self retaining retractors are not effective or applicable in such setting because they require additional bracing to keep them in place.

It is an object of the current invention to provide a surgical retractor which maintains the incision in an open position.

It is another object of the current invention to provide a retractor that is light and be stably supported by the soft tissue of the surgical field that is being retracted without additional bracing means.

### SUMMARY OF THE INVENTION

The present invention is a circumferential retractor for maintaining an incision in an open position. The retractor provides an aperture as delineated by its inner periphery for which a surgeon can operate through. It is generally oval in shape with flanges to keep the retracted tissues within and outside of its outer periphery. The retractor has a rigid configuration. For it to retract incisions of different length, the surgeon selects the appropriately sized retractor according to the incision size. The retractor is maintained in position by opposing forces of the soft tissue being retracted whereby no additional mechanism is necessary to keep it in place.

### BRIEF DESCRIPTION OF THE DRAWING

FIG. 1 is a perspective view of an embodiment of the present invention in a wrist incision;

FIG. 2 is an oblique view of the present invention.

### DETAILED DESCRIPTION OF THE INVENTION

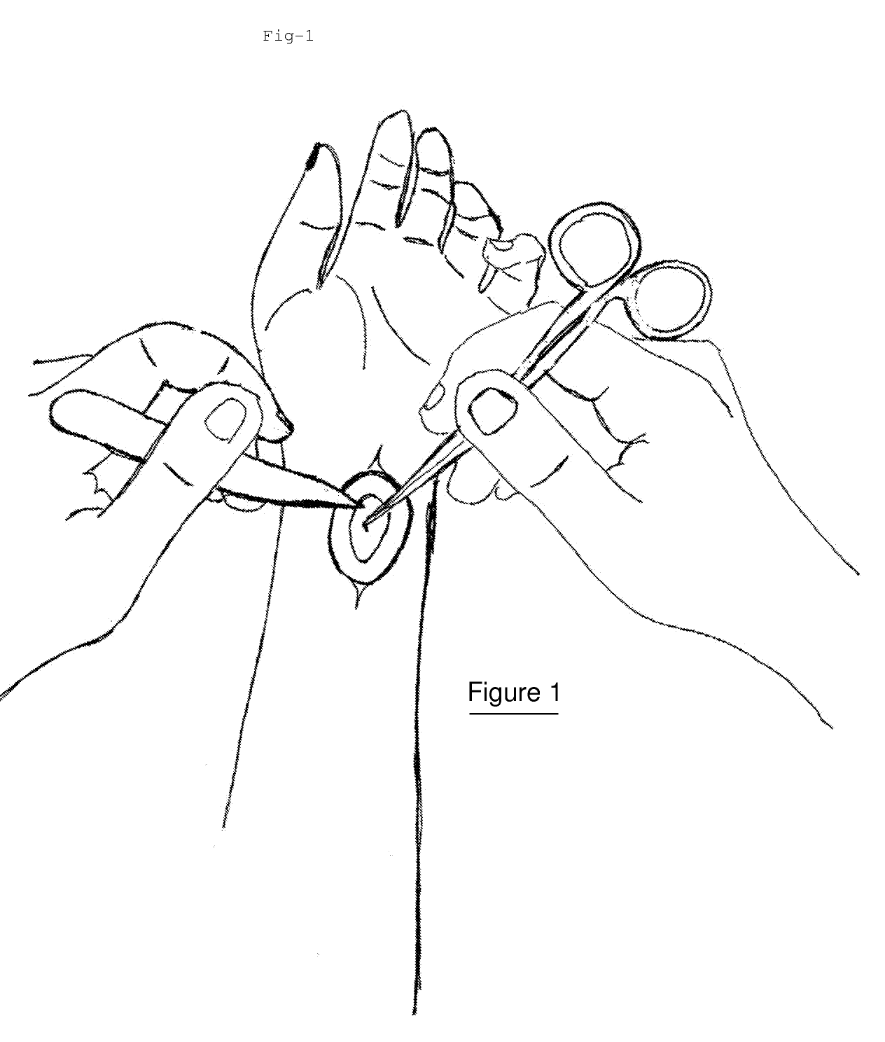

Referring to FIG. 1, there is shown the retractor in place in a wrist incision. The device is generally of an elliptical construction maintaining the incision in an open position. The retractor as shown is at least large enough such that instruments or a surgeon's fingers can be placed through its aperture.

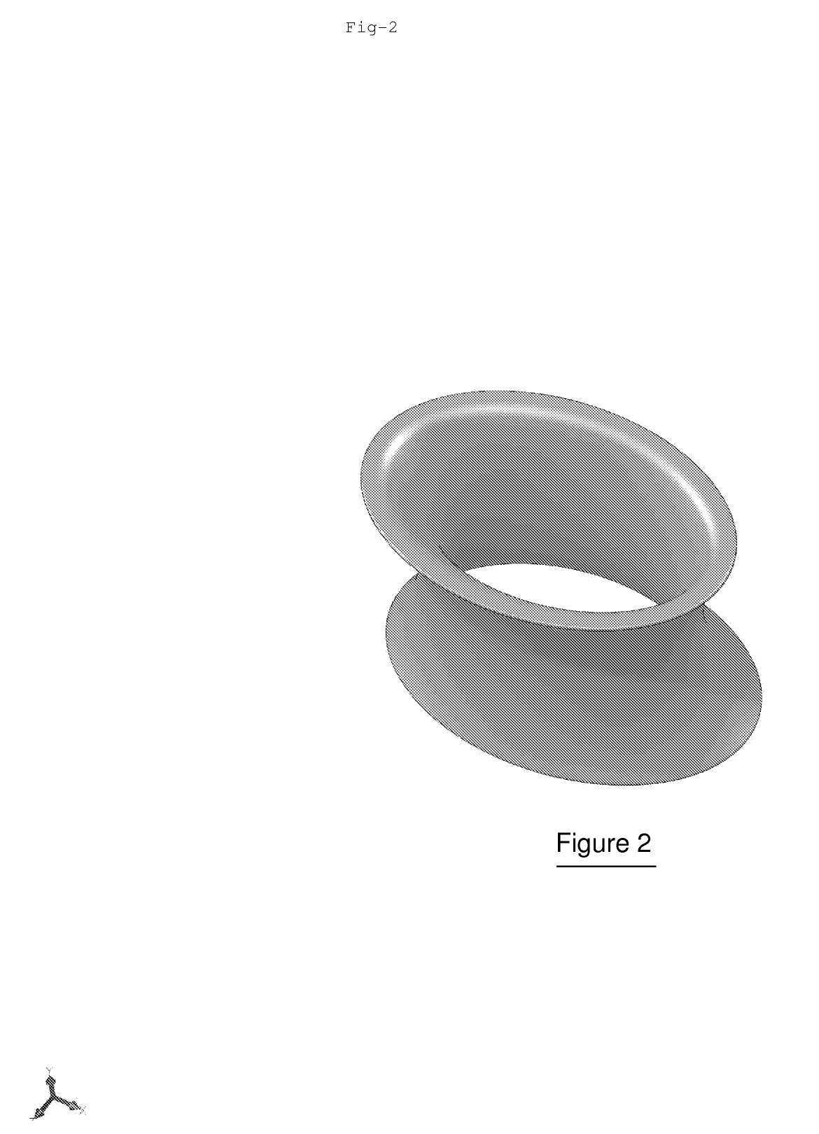

Referring to FIG. 2, the retractor is narrower in its mid-section to allow the flanges on either end to create a catchment area by which soft tissue can be retracted. The retractor has no moving parts and no projections so as to minimize soft tissue trauma during soft tissue retraction.

To achieve retraction of soft tissues in surgical fields of different incision sizes, the surgeon selects an appropriate size of the retractor that the incision would allow. Once placed the opposing forces of the soft tissue being retracted stabilizes and maintains the retractor in place. The thickness between the inner and outer periphery may vary depending on the rigidity or stiffness of the surgical field being retracted.

The present invention while simple in concept achieves some notable advantages over conventional retractors. Use of differing sizes of the device according to the incision size allows it to retract tissue and maintain its position without complex mechanical means beyond its shape. This main feature leads to a self-retaining retractor that is light and can be stably supported by opposing forces of the soft tissue being retracted.

The foregoing disclosure and description of the invention is illustrative and explanatory thereof. Modifications in the details of its construction can be made within the scope of the appended claims without departing from the spirit and scope of the invention. The present invention should only be limited by the following claims and their legal equivalents.

1. A device for holding open a surgical incision comprising:
an elliptical aperture with a rigid spline wall that defines an interior compartment with an exterior surface, whereby tissues are engaged and prevented from collapsing into said aperture.

2. The device as recited in claim 1 has no moving parts, relying solely on the shape of its wall and flange design to maintain retracted tissues within and outside of its outer periphery.

3. A method of retracting tissue comprising of selecting an appropriate size for said device in claim 1 to be engaged by a substantial portion of said incision, and positioning said device to retract tissue and maintains the incision in an open position allowing surgical access through said aperture.

4. The device as recited in claim 1 elliptical shape changes from top to bottom following a specific spline. The ellipse is smaller in the center of the device than top and bottom. This provides stability inside the incision.

5. The device as recited in claim 1 inside edges on top and bottom are filleted facilitating the ease of use of surgical instruments.

6. The device as recited in claim 1 outside edges on top and bottom are filleted reducing tissue damage.

7. The device as recited in claim 1 elliptical shape and size reduces tissue trauma.
